(19) United States
(12) Patent Application Publication
(10) Pub. No.: US 2010/0047363 A1
(43) Pub. Date: Feb. 25, 2010

(54) NUTRITIONAL SUPPLEMENT FOR PATIENTS WITH CHRONIC KIDNEY DISEASE

(76) Inventor: John Wigneswaran, Dayton, OH (US)

Correspondence Address: THOMPSON HINE L.L.P.;Intellectual Property Group, P.O. BOX 8801, DAYTON, OH, 45401-8801, US

(21) Appl. No.: 12/357,602

(22) Filed: Jan. 22, 2009

Related U.S. Application Data
(60) Provisional application No. 61/112,297, filed on Nov. 7, 2008.

(51) Int. Cl. A61K 33/34; A61K 31/51; A61P 13/12

(52) U.S. Cl. 424/638; 514/276

(57) ABSTRACT

A nutritional supplement for patients having chronic kidney disease. The nutritional supplement comprises:

| | Approximate | |
|---|---|---|
| | Amount | |
| Vitamin C | 60-500 | |
| Vitamin D (as cholecalciferol) | 400-10,000 | IU |
| Thiamin (Vitamin B1) | 1-50 | mg |
| Riboflavin (Vitamin B2) | 1.1-10 | mg |
| Vitamin B6 | 2-50 | mg |
| Folic Acid | 0.4-5 | mg |

wherein the supplement is further characterized in that it does not contain amounts of Vitamin A, Vitamin K, potassium, phosphorus, chromium, or vanadium that would be detrimental to a patient having CKD. In a particular embodiment, the supplement also contains omega fatty acids.

### CROSS REFERENCE TO RELATED APPLICATION

This application claims the benefit of U.S. Provisional Application Ser. No. 61/112,297 filed Nov. 7, 2008, the contents of which are hereby incorporated by reference.

### FIELD OF THE INVENTION

The present invention is directed to a nutritional supplement for individuals suffering from chronic kidney disease (hereinafter “CKD”). CKD is a medical condition characterized by a gradual and permanent loss of kidney function. People with CKD have unique nutritional requirements as a result of alterations in absorption, metabolism, and excretion of many vitamins, minerals, and electrolytes. As a result, the intake of certain nutrients must be avoided or reduced, while higher doses of other nutrients are required to maintain health.

### DESCRIPTION OF THE INVENTION

The present invention provides a nutritional supplement for people with CKD that in one embodiment comprises the following agents in the following approximate amounts:

| Vitamin/mineral | Range | |
|---|---|---|
| Vitamin C | 60-500 | mg |
| Vitamin D (as cholecalciferol) | 400-10,000 | IU |
| Vitamin E (as d-alpha tocopherol) | 0-800 | IU |
| Thiamin (Vitamin B1) | 1-50 | mg |
| Riboflavin (Vitamin B2) | 1.1-10 | mg |
| Niacin (as Niacinamide) | 0-1,000 | mg |
| Vitamin B6 | 2-50 | mg |
| Folic Acid | 0.4-5 | mg |
| Vitamin B12 | 0-1,000 | mcg |
| Biotin | 0-300 | mcg |
| Pantothenic Acid | 0-10 | mg |
| Calcium (elemental) | 0-1,200 | mg |
| Iron | 0-150 | mg |
| Zinc | 0-50 | mg |
| Selenium | 0-400 | mcg |
| Copper | 0-10,000 | mcg |
| Omega-3 Fatty Acids | 0-4,000 | mg |

The supplement is also characterized by what it does not contain. Certain agents that are not desirable for CKD patients when administered or consumed on a regular or a daily basis are not present. In one embodiment the supplement does not contain potassium, phosphorus, chromium, or vanadium, and, in one embodiment it also does not contain Vitamin A in amounts that would be detrimental to a patient having CKD. For convenience, in one embodiment, the supplement does not contain any of these agents. We are also not including Vitamin K, as patients with CKD generally do not have evidence of deficiency. In addition, patients with CKD often take Warfarin for other related medical conditions, and Vitamin K can antagonize the action of that medication resulting in harm. While it is desirable to eliminate the foregoing nutrients entirely, those skilled in the art will recognize that the addition of amounts of these excluded agents that are too small to harm the patient will not avoid infringement of this invention. While in one embodiment, the supplement contains no Vitamin A, up to about 0.7 mg RE may be tolerated and formulations including such amounts are not outside the scope of the invention. Similarly, up to about 0.02 mg of chromium may be tolerated. In another embodiment the supplement does not contain calcium.

The supplement preferably includes specific vitamins and minerals.

Vitamin D: Vitamin D deficiency is common in patients with CKD for several reasons including reduced endogenous synthesis of D3, lower calcium intake, and urinary loss of Vitamin D and binding proteins with nephrotic proteinuria. Though the RDI of Vitamin D is currently 400 IU, a number of studies have shown that doses in excess of 800 IU correlate with reduced rates of fractures, falls, and cancer. KDOQI guidelines recommend 800 IU to prevent deficiency in patients over the age of 60, but doses as high as 1,000 IU daily have been recommended in the field for patients with CKD to prevent deficiency. In one embodiment, the supplement contains about 400 to 10,000 IU. In another embodiment it contains about 400 to 2,000 IU, or in another embodiment, about 700 to 1,000 IU to prevent deficiency based on the above guidelines and evidence.

Vitamin E: In one embodiment up to about 800 IU is included. In another embodiment, the supplement contains up to about 400 IU. In another embodiment, the supplement contains up to about 100 IU.

Vitamin C: In high doses, Vitamin C can result in increased plasma levels of oxalate, thereby increasing the risk of nephrolithiasis and intrarenal deposition of calcium oxalate resulting in renal failure. Leading multivitamin supplements often have amounts of Vitamin C in excess of the RDI. In one embodiment Vitamin C is limited to not more than about 500 mg. In another embodiment, the supplement contains no more than about 200 mg, and in still another embodiment it contains about 60 to 90 mg Vitamin C.

Riboflavin (Vitamin B2) and Pyridoxine (Vitamin B6): Though higher than the RDI, in accordance with the embodiment illustrated in the table, doses of Vitamin B6 can run up to about 50 mg and doses of Vitamin B2 can run up to 10 mg. In a particular embodiment doses of vitamin B1 will be about 1 to 10 mg. In one embodiment, the dose of Vitamin B2 will be about 1 to 4 mg. In one embodiment, the dose of Vitamin B6 will be about 2 to 10 mg.

Omega-3 Fatty Acids (DHA+EPA): It is increasingly recognized that CKD is an independent risk factor for cardiovascular morbidity and mortality. This is as a result of both traditional risk factors (such as hypertension and hyperlipidemia) and non-traditional risk factors (such as abnormal vascular calcification). In addition, CKD is felt to cause an inflammatory milieu which leads to accelerated atherosclerosis. Omega-3 fatty acids, specifically docosahexaenoic acid (DHA) and eicosapentaenoic acid (EPA), have been shown in multiple prospective clinical trials and observational studies to significantly reduce the risk of cardiovascular morbidity and mortality (rev Lee et al. Mayo Clin Proc 2008; 83 (3):324). Omega-3 fatty acids also have been reported to have beneficial effects on blood pressure and hyperlipidemia and may have anti-inflammatory properties that lead to a reduction in atherosclerosis. The American Heart Association has recommended intake of omega-3 fatty acids as part of a comprehensive strategy to prevent cardiovascular events (American Heart Association Clinical Guidelines, 2002). While certain embodiments of the invention may employ up to 4,000 mg of these fatty acids, in another embodiment, no fish oil is present. When present about 250 mg appears sufficient, but the skilled artisan can readily increase this. In a particular embodiment about 100 to 500 mg is used. Additionally, while it is convenient for the patient if the fish oil is contained in the same oral dosage unit (e.g., capsule, tablet, caplet, etc.) of the supplement, it is also within the scope of the invention to co-administer the fish oil in the form of a separate capsule.

As described herein, by omitting certain nutrients, adjusting the doses of others, and adding nutrients typically not found in multivitamins, the formulations disclosed herein represent approach nutritional supplementation in chronic kidney disease.

The invention is illustrated by the following non-limiting examples.

### EXAMPLE A

| Ingredient | Amount | |
|---|---|---|
| Folic Acid | 1 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 250 | mg |
| Fish Oil (DHA + EPA) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 30 | mg |
| Zinc | 8 | mg |
| Copper | 900 | mcg |
| Selenium | 200 | mcg |

### EXAMPLE B

| Ingredient | Amount | |
|---|---|---|
| Folic Acid | 0.8 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 100 | mg |
| Fish Oil (DHA + EPA) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 20 | mg |
| Zinc | 8 | mg |
| Copper | 900 | mcg |
| Selenium | 55 | mcg |

### EXAMPLE C

| Ingredient | Amount | |
|---|---|---|
| Folic Acid | 1 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 30 | mg |
| Zinc | 8 | mg |
| Copper | 900 | mcg |
| Selenium | 200 | mcg |

### EXAMPLE D

| Ingredient | Amount | |
|---|---|---|
| Folic Acid | 0.8 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 100 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 20 | mg |
| Zinc | 8 | mg |
| Copper | 900 | mcg |
| Selenium | 55 | mcg |

In another embodiment of the invention, there is no fish oil, no iron, no calcium and no Vitamin E in the formulation. Example E is one example of this embodiment of the invention.

### EXAMPLE E

| Ingredient | Amount | |
|---|---|---|
| Folic Acid | 0.8 | mg |
| B6 | 10 | mg |
| B12 | 2.4 | mcg |
| Iron | NONE | |
| Vitamin C | 90 | mg |
| Vitamin D (cholecalciferol) | 200 | IU |
| Calcium | NONE | |
| Vitamin E | NONE | |
| Thiamine | 1.5 | mg |
| Riboflavin | 2 | mg |
| Biotin | 30 | mcg |
| Pantothenic Acid | 5 | mg |
| Niacin | 20 | mg |
| Zinc | 8 | mg |
| Copper | 900 | mcg |
| Selenium | 55 | mcg |

While the invention has been described in detail and by reference to specific examples and embodiments, those skilled in the art will recognize that deviations and variations are possible without departing from the scope of the invention as defined by the following claims.

What is claimed is:

1. A nutritional supplement comprising:

| | Approximate | |
|---|---|---|
| | Amount | |
| Vitamin C | 60-500 | |
| Vitamin D (as cholecalciferol) | 400-10,000 | IU |
| Thiamin (Vitamin B1) | 1-50 | mg |
| Riboflavin (Vitamin B2) | 1.1-10 | mg |
| Vitamin B6 | 2-50 | mg |
| Folic Acid | 0.4-5 | mg |

wherein the supplement is further characterized in that it does not contain amounts of Vitamin A, Vitamin K, potassium, phosphorus, chromium, or vanadium that would be detrimental to a patient having CKD.

2. The supplement of claim 1 wherein the supplement further comprises omega fatty acids in an amount up to about 4,000 mg (calculated based on the amount of DHA and EPA).

3. The supplement of claim 2 wherein the amount of the omega fatty acids is about 100 to 500 mg.

4. The supplement of claim 2 wherein the supplement includes Vitamin A in an amount up to about 0.7 mg RE.

5. The supplement of claim 2 wherein the supplement contains about 2 to 10 mg Vitamin B6.

6. The supplement of claim 2 wherein the supplement contains about 1 to 10 mg Vitamin B1.

7. The supplement of claim 2 wherein the supplement contains about 1 to 4 mg Vitamin B2.

8. The supplement of claim 2 wherein the supplement contains about 400 to 2,000 IU of Vitamin D.

9. The supplement of claim 2 wherein the omega fatty acids are present in the same oral dosage unit as the balance of the supplement.

10. The supplement of claim 2 containing the following agents in the approximate amounts indicated:

| | | |
|---|---|---|
| Vitamin E (as d-alpha | 0-800 | IU |
| tocopherol) | | |
| Niacin (as Niacinamide) | 0-1,000 | mg |
| Folic Acid | 0.4-5 | mg |
| Vitamin B12 | 0-1,000 | mcg |
| Biotin | 0-300 | mcg |
| Pantothenic Acid | 0-10 | mg |
| Calcium (elemental) | 0-1,200 | mg |
| Iron | 0-150 | mg |
| Zinc | 0-50 | mg |
| Selenium | 0-400 | mcg |
| Copper | 0-10,000 | mcg |

11. The supplement of claim 2 containing the following agents in the approximate amounts indicated:

| | | |
|---|---|---|
| Folic Acid | 1 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 250 | mg |
| Fish Oil (DHA + EPA) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 30 | mg |
| Zinc | 8 | mg |
| Copper | 900 | mcg |
| Selenium | 200 | mcg |

12. The supplement of claim 2 containing the following agents in the approximate amounts indicated:

| | | |
|---|---|---|
| Folic Acid | 0.8 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 100 | mg |
| Fish Oil (DHA + EPA) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 20 | mg |
| Zinc | 8 | mg |
| Copper | 900 | mcg |
| Selenium | 55 | mcg |

13. The supplement of claim 2 containing the following agents in the approximate amounts indicated:

| | | |
|---|---|---|
| Folic Acid | 1 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 250 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 30 | mg |
| Zinc | 8 | mg |
| Copper | 900 | mcg |
| Selenium | 200 | mcg |

14. The supplement of claim 2 containing the following agents in the approximate amounts indicated:

| | | |
|---|---|---|
| Folic Acid | 0.8 | mg |
| B6 | 5 | mg |
| B12 | 2.4 | mcg |
| Iron | 8 | mg (elemental) |
| Vitamin C | 60 | mg |
| Vitamin D (cholecalciferol) | 800 | IU |
| Calcium (elemental) | 100 | mg |
| Vitamin E | 10 | IU |
| Thiamine | 1.5 | mg |
| Riboflavin | 2 | mg |
| Biotin | 30 | mcg |
| Pantothenic Acid | 5 | mg |
| Niacin | 20 | mg |
| Zinc | 8 | mg |
| Copper | 900 | mcg |
| Selenium | 55 | mcg |

15. The supplement of claim 9 wherein the supplement is essentially free of Vitamin A, Vitamin K, potassium, chromium, vanadium and calcium.

16. A method for preventing or delaying the progression of cardiovascular disease in a patient having CKD which comprises administering to a patient diagnosed with CKD, on a daily basis, the nutritional supplement defined in claim 1.

17. The method of claim 16 wherein the supplement is administered once daily.

18. The method of claim 16 wherein the supplement further comprises omega fatty acids in an amount up to about 4,000 mg.

19. The method of claim 18 wherein the supplement is essentially free of Vitamin A, Vitamin K, potassium, chromium, vanadium and calcium.

20. The method of claim 19 wherein the omega fatty acids are contained in the same oral dosage unit as the balance of the supplement.
